(19) United States
(12) Patent Application Publication
(10) Pub. No.: US 2026/0357240 A1
(43) Pub. Date: Nov. 12, 2026

(54) COMPOSITION AND METHOD OF MANUFACTURING OF AN IMMOBILIZED LIPASE ON SILICA

(71) Applicant: ADVANCED MATERIALS & CATALYSTS LLC., Conshohocken, PA (US)

(72) Inventors: Caio Cesar DE CARVALHO, Conshohocken, PA (US); Ye LIU, Conshohocken, PA (US); Parag Rasiklal SHAH, Lafayette Hill, PA (US)

(21) Appl. No.: 19/670,871

(22) Filed: May 7, 2026

Related U.S. Application Data
(60) Provisional application No. 63/801,628, filed on May 7, 2025.

(51) Int. Cl. C12N 11/14; C12N 9/20

(52) U.S. Cl. CPC C12N 11/14; C12N 9/20; C12Y 301/01003

(57) ABSTRACT

The disclosure provides compositions comprising an immobilized enzyme and carrier as well as methods of making and uses thereof. In an embodiment, the compositions comprise an immobilized lipase B from Candida antartica (CalB) enzyme which is stable over multiple reuse cycles. In an embodiment, the carrier is a porous silica gel that is functionalized by methacryl group using silanes. The disclosure further provides method for immobilizing an enzyme using the disclosed composition, as well as methods of performing an enzymatic reaction using the disclosed compositions.

This application claims the benefit of priority of U.S. Provisional Application No. 63/801,628, filed May 7, 2025, the contents of which are incorporated by reference herein in their entirety.

### TECHNICAL FIELD

Disclosed herein is a novel composition comprising an immobilized lipase B from Candida antartica (CalB) enzyme which is stable over multiple reuse cycles. In an embodiment, the carrier is a porous silica gel that is functionalized by methacryl group using silanes. The present disclosure is directed to compositions, methods of manufacture, and applications for the disclosed composition.

### BACKGROUND

The enzyme lipase B Candida antartica (CalB) is among the most stable commercialized enzymes that have been used in a wide range of reactions like hydrolysis of esters, transesterification and esterification of acids, and acylation of amines. Commercial applications include biodiesel production, regioselective reactions for pharmaceuticals and agrochemicals, polymer degradation, etc. CalB is commercially available as a free enzyme (e.g. Lipozyme® CalB-L or Sustine® 111) and an immobilized enzyme (e.g. Novozym® 435 or Sustine® 110 IM). A free enzyme refers to an enzyme that is in its natural, soluble form, freely dispersed in the reaction medium. These enzymes are not attached to any support or matrix, allowing them to move freely and interact with substrates. Free enzymes are typically used in homogeneous reactions where the enzyme and substrate are in the same phase. However, they can be less stable and more difficult to recover and reuse after the reaction. An immobilized enzyme is an enzyme that is physically confined or localized in a certain defined region of space, often attached to or trapped within a solid support or matrix, while retaining catalytic activity. This immobilization can be achieved through various methods such as adsorption, covalent bonding, entrapment, or encapsulation. Immobilized enzymes offer several advantages, including enhanced stability, ease of separation from reaction mixtures, and the potential for reuse in multiple reaction cycles. They are widely used in industrial processes due to these benefits.

Novozym® 435 (N435), now rebranded as Sustine® 110 IM, is CalB immobilized on an acrylic polymer resin Lewatit® VP OC1600 via interfacial interaction. While N435 has found commercial success, it presents drawbacks related to enzyme leaching and therefore activity loss, support solubility in organic media, mechanical fragility, and high cost. Additionally, disposing of the spent catalyst in an environmentally responsible manner is challenging because the polymer resins are non-biodegradable.

Numerous studies have explored methods to enhance the performance of immobilized CalB. Among these, several have employed silica functionalized with the methacryl moiety as a support material. For example, Weltz et. al., ACS Catal. 2019, 9, 4992-5001 discloses immobilized CalB on poly(sulfobetaine methacrylate) and poly(ethylene glycol) methacrylate brushes grown on silica particles. Wunschik et. al., Appl. Biochem. Biotechnol. 2022, 194, 3384-3399 discloses immobilized CalB on a core-shell nano-particle consisting of silica nanoparticle core and shell of polymethylmethacrylate polymer brushes (MW~31000±600 g/mol). Li et. al., Int. J. Biol. Macromol. 2018, 120, 886-895 discloses immobilized CalB on ordered mesoporous silica (SBA-15) functionalized separately with various functional groups using silanes, and no loss of activity was observed for SBA-15 functionalized with propyl methacrylate group.

The synthesis of immobilized CalB compositions involving polymer brushes requires multi-step reactions to grow the brushes, which inherently increases costs. Similarly, SBA-15 type mesoporous ordered silicas involve complex synthesis procedures, potentially rendering the final product cost-prohibitive for many applications. Additionally, there is a difference in pore structures of SBA-15 and amorphous silica gels that are manufactured by the process described herein. SBA-15 silicas feature cylindrical unidirectional mesopores with a hexagonal ordering. The SBA-15 mesopores exhibit a narrow pore size distribution and are connected by micropores. Conversely, the silica gels provided by the process described herein possess mesopores with a broader pore size distribution that are interconnected in three dimensions. This structural distinction may offer advantages in terms of mass transfer for enzymes being immobilized, as well as for the reactants and products involved in enzymatic catalysis.

### SUMMARY

Consistent with some disclosed embodiments there is described an immobilized CalB enzyme that surpasses the current state-of-the-art technologies. The enzyme is supported on a non-ordered amorphous silica gel, which is functionalized with a silane containing the methacryloxypropyl group. Application testing with the hydrolysis reaction has demonstrated that the disclosed product maintains its activity over 10 reuse cycles, whereas the benchmark (Sustine® 110 IM) loses ~⁴⁰% of its activity after just one cycle. Furthermore, the disclosed silica-based product is mechanically robust, insoluble in most organic media, and can be disposed responsibly. Made from readily available raw materials, the disclosed silica is scalable to large volumes and is more cost-effective than ordered mesoporous silicas like SBA-15.

In one embodiment, there is disclosed a method for immobilizing an enzyme that comprises a non-ordered amorphous silica having a silica surface that is functionalized with a methacryl group, and a lipase immobilized on the silica surface that is functionalized with the methacryl group. The method comprises: functionalizing a silica gel using an impregnation technique with a methacryl group precursor; preparing a solution comprising the enzyme stabilized in a buffer; optionally pretreating the functionalized silica; and adding the solution to the functionalized silica and mixing to form an immobilized enzyme.

Consistent with some disclosed embodiments there is described a method of performing an enzymatic reaction, In one embodiment, the method comprises: contacting a substrate with an immobilized enzyme comprising: non-ordered amorphous silica having a silica surface that is functionalized with a methacryl group; and a lipase immobilized on the silica surface that is functionalized with the methacryl group. In one embodiment, contacting is performed under conditions effective to catalyze conversion of the substrate to a reaction product.

### BRIEF DESCRIPTION OF THE FIGURES

The accompanying drawings, which are incorporated in and constitute a part of this specification, illustrate some disclosed embodiments and, together with the description, serve to explain the disclosed embodiments. The particulars shown are by way of example and for purposes of illustrative discussion of embodiments of the present disclosure. The description taken with the drawings makes apparent to those skilled in the art how embodiments of the present disclosure may be practiced.

FIG. 1 depicts a chart showing the influence of temperature and time on enzyme uptake.

FIG. 2 compares the activity of the immobilized enzyme of the current disclosure and the immobilized enzyme Sustine® 110IM over ten reaction cycles.

FIG. 3 compares the transesterification activity of the disclosed immobilized CalB to the transesterification activity of Sustine® 110 IM.

FIG. 4A depicts the immobilized enzyme of the present disclosure before use (left) and after use (right).

FIG. 4B depicts the immobilized enzyme Sustine® 110IM before use (left) and after use (right).

### DETAILED DESCRIPTION

### Definitions

Unless otherwise indicated, the terms “comprising,” “including,” and “containing” are used herein in an open-ended sense and do not exclude the presence of additional, unrecited elements or method steps. All numerical ranges disclosed herein include all values and subranges encompassed therein.

The immobilized enzyme compositions described herein—including immobilized Candida antarctica lipase B (CalB) on silica—may optionally comprise one or more additional components that function as additives, excipients, or both. Such components may be present alone or in any combination, and in any amount effective to achieve desired performance characteristics, functional attributes, or physicochemical properties of the immobilized enzyme composition.

As used herein, an “additive” is a compound or material included primarily to improve, enhance, or modify functional performance of the immobilized enzyme, including but not limited to catalytic activity, stability, thermal resistance, chemical resistance, operational robustness, or storage stability. An “excipient” as used herein is a compound or material included primarily to facilitate formulation, handling, manufacture, processing, storage, or use of the immobilized enzyme composition, without necessarily exerting a direct effect on catalytic activity, although incidental functional effects are not excluded. A given component may function as an additive, an excipient, or both, depending on formulation context, concentration, method of incorporation, or intended application, and all such dual function components are expressly encompassed by the present disclosure.

The term “average particle size” used herein refers to the volume mean size of particles in a particulate material, typically reported as an equivalent spherical diameter, as determined from a particle size distribution measured by a specified method (e.g., laser diffraction, sieve analysis, or microscopy/image analysis).

The term “surface area” used herein refers to the total area of a material's external surface plus any accessible internal pore surfaces. In porous solids (e.g., silica), it is commonly reported as specific surface area (surface area per unit mass, e.g., m²/g) and is typically determined by gas adsorption methods (e.g., BET analysis).

The term “pore volume” used herein refers to the total volume of pores within a porous material, typically reported on a mass-normalized basis (e.g., cc/g), as determined from a pore volume measurement method such as gas adsorption (e.g., nitrogen adsorption/desorption), mercury intrusion porosimetry or by infiltration of pores with a liquid and represents the cumulative volume of accessible internal void spaces within the material.

The term “average pore diameter” (also referred to as “average pore size”) as used herein refers to the mean size of the pores in a porous material, typically reported as an equivalent pore diameter. The average pore diameter is calculated from the surface area and pore volume using the following equation, assuming cylindrical pore geometry:

$$Averge pore diameter ( Å ) = \frac{40000 \times PV ( \frac{cc}{g} )}{SA ( \frac{{m}^{2}}{g} )}$$

The term “non-ordered amorphous silica” as used herein, refers to a silica material comprising a three-dimensional silicon-oxygen network that lacks both (i) long-range atomic periodicity characteristic of crystalline silica phases; and (ii) long-range mesostructural ordering or periodic pore arrangement characteristic of templated ordered mesoporous silicas. The non-ordered amorphous silica is further characterized by a stochastically formed, non-periodic pore network comprising interconnected and tortuous voids having a heterogeneous pore size distribution and is formed in the absence of structure-directing agents that induce supramolecular pore ordering. Non-ordered amorphous silica is distinguished from crystalline silica in that it does not exhibit long-range atomic lattice order and lacks sharp diffraction peaks in wide-angle X-ray diffraction analyses corresponding to crystalline phases such as quartz, cristobalite, or tridymite. Non-ordered amorphous silica is further distinguished from ordered mesoporous silicas, including SBA-type, MCM-type, and KIT-type materials, in that it does not exhibit periodic pore symmetry, long-range mesoscopic ordering, or uniform pore channels arranged in defined geometries.

### Analysis Methods:

Particle size and particle size distribution may be measured by various techniques known in the art, including laser diffraction, dynamic image analysis, and sieve analysis. Regardless of the measurement technique employed, particle size values are reported herein as an equivalent spherical diameter derived from the measured particle size distribution. In laser diffraction methods, for example using and apparatus known as Malvern Mastersizer (made the Malvern Panalytical from Malvern, Worcestershire, United Kingdom), the distribution is calculated from the angular variation in intensity of light scattered by dispersed particles using appropriate optical models, such as Mie scattering theory. In dynamic image analysis methods, for example using a Camsizer® system (made by Retsch Technology GmbH, Haan, Germany), individual particle images are captured and analyzed to determine characteristic particle dimensions (e.g. area equivalent diameters), which are converted into an equivalent spherical diameter distribution. In sieve analysis, particles are mechanically classified into size fractions based on passage through a series of sieves of defined mesh size, and the distribution is determined from the mass percentage retained on or passing through each sieve, with representative particle diameters assigned to each fraction. Unless otherwise stated, particle size distributions obtained by any of these methods are converted, where appropriate, to a volume based particle size distribution, and the average particle size refers to the volume mean particle diameter calculated therefrom.

The surface area and pore volume were measured by Nitrogen Porosimetry using an Autosorb-6 Testing Unit from Quantachrome Corporation (now Anton Paar GmbH) or a Micromertics ASAP 2460 Unit. Samples were first degassed at 350 °C for at least 4 hours on the Autosorb-6 Degassing Unit. A multipoint surface area is calculated using the BET theory taking data points in the P/P₀ range 0.05 to 0.30.

For silicas comprising pores with diameters smaller than 50 nm, pore volume is determined from gas adsorption measurements, with the pore volume recorded at a relative pressure (P/P₀) of 0.984 on the desorption branch of the isotherm.

For silicas comprising pores with diameters greater than 50 nm, pore volume may be determined by mercury intrusion porosimetry. Alternatively, pore volume may be determined by infiltrating the pores with a liquid, such as water or alcohol, and measuring the amount of liquid required to saturate the pore structure. Saturation may be identified by a change in the flow properties of the silica powder, for example when a free-flowing powder exhibits reduced flowability or ceases to flow freely due to the presence of liquid on the particle surfaces.

The average pore diameter is then calculated using the following equation, assuming cylindrical pore geometry:

$$Averge pore diameter ( Å ) = \frac{40000 \times PV ( \frac{cc}{g} )}{SA ( \frac{{m}^{2}}{g} )}$$

The mean pore diameter of the pore structure of a silica support particle is directly proportional to the pore volume and inversely proportional to the pore surface area. Accordingly, it is possible for two carrier particles to exhibit the same mean pore diameter while differing significantly in pore volume and surface area. For example, a first support particle may possess a relatively low pore volume and low surface area, whereas a second support particle may possess a relatively high pore volume and high surface area, notwithstanding that both have the same mean pore diameter.

It is desirable that the pore diameter of the pores constituting the pore structure be sufficiently large to permit, first, ingress of an enzyme into the pores for immobilization therein, and second, ingress of reactant molecules to interact with the immobilized enzyme to form a reaction product. The pore structure should further allow for efficient diffusion of the reaction products out of the pores.

### Compositions:

The disclosure provides compositions comprising an immobilized enzyme. In some embodiments, the immobilized enzyme comprises non-ordered amorphous silica having a silica surface that is functionalized with a methacryl group. In some embodiments, the immobilized enzyme comprises a lipase immobilized on the silica surface that is functionalized with the methacryl group.

In some embodiments, the disclosure provides compositions comprising an immobilized enzyme, wherein the immobilized enzyme comprises amorphous silica having an average particle size ranging from 10 μm to 10 mm. In some embodiments, the amorphous silica has an average particle size ranging from 100 μm to 1 mm. In some embodiments, the amorphous silica has an average particle size ranging from 100 μm to 300 μm. In some embodiments, the amorphous silica has an average particle size of about 200 μm. In some embodiments, the amorphous silica has an average particle size ranging from 300 μm to 700 μm. In some embodiments, the amorphous silica has an average particle size of about 500 μm.

In some embodiments, the disclosure provides compositions comprising an immobilized enzyme, wherein the immobilized enzyme comprises amorphous silica having an average pore diameter ranging from 15-150 nm. In some embodiments, the amorphous silica has an average pore diameter ranging from 20-100 nm. In some embodiments, the amorphous silica has an average pore diameter ranging from 30-80 nm. In some embodiments, the amorphous silica has an average pore diameter ranging from 30-60 nm. In some embodiments, the amorphous silica has an average pore diameter ranging from 40-60 nm. In some embodiments, the amorphous silica has an average pore diameter of about 30 nm, about 35 nm, about 40 nm, about 45 nm, about 50 nm, about 55 nm, about 60 nm, about 65 nm, about 70 nm, about 75 nm, or about 80 nm. In some embodiments, the amorphous silica has an average pore diameter of about 35 nm. In some embodiments, the amorphous silica has an average pore diameter of about 50 nm.

In some embodiments, the disclosure provides compositions comprising an immobilized enzyme, wherein the immobilized enzyme comprises amorphous silica having a surface area in the range of 10 m²/g to 500 m²/g. In some embodiments, the amorphous silica has a surface area in the range of 10 m²/g to 200 m²/g. In some embodiments, the amorphous silica has a surface area in the range of 30 m²/g to 150 m²/g. In some embodiments, the amorphous silica has a surface area in the range of 60 m²/g to 150 m²/g. In some embodiments, the amorphous silica has a surface area in the range of 60 m²/g to 120 m²/g. In some embodiments, the amorphous silica has a surface area of about 60 m²/g, about 70 m²/g, about 80 m²/g, about 90 m²/g, about 100 m²/g, about 110 m²/g, about 120 m²/g, about 130 m²/g, about 140 m²/g, or about 150 m²/g. In some embodiments, the amorphous silica has a surface area of about 110 m²/g.

In some embodiments, the disclosure provides compositions comprising an immobilized enzyme, wherein the immobilized enzyme comprises amorphous silica having a pore volume ranging from 0.1 cc/g to 5.0 cc/g. In some embodiments, the amorphous silica has a pore volume ranging from 0.1 cc/g to 3.5 cc/g. In some embodiments, the amorphous silica has a pore volume ranging from 0.5 cc/g to 2.0 cc/g. In some embodiments, the amorphous silica has a pore volume ranging from 0.8 cc/g to 1.2 cc/g. In some embodiments, the amorphous silica has a pore volume of about 1.0 cc/g.

In some embodiments, the disclosure provides compositions comprising an immobilized enzyme, wherein the immobilized enzyme comprises amorphous silica having a hydroxyl group concentration ranging from 0.1 OH/nm²-5 OH/nm². In some embodiments, the amorphous silica has a hydroxyl group concentration ranging from 1 OH/nm²-5 OH/nm². In some embodiments, the amorphous silica has a hydroxyl group concentration ranging from 2 OH/nm²-4 OH/nm². In some embodiments, the amorphous silica has a hydroxyl group concentration of about 1 OH/nm², about 2 OH/nm², about 3 OH/nm², about 4 OH/nm², or about 5 OH/nm². In some embodiments, the amorphous silica has a hydroxyl group concentration of about 3 OH/nm².

In some embodiments, the disclosure provides compositions comprising an immobilized enzyme, wherein the immobilized enzyme comprises non-ordered amorphous silica functionalized with a methacryl group. In some embodiments, the methacryl group is derived from a silane precursor.

In some embodiments, the methacryl group is derived from a methacrylate-functional silane precursor. In some embodiments, the methacrylate-functional silane precursor comprises at least one methacrylate group and at least one hydrolyzable silyl group. In some embodiments, the methacrylate-functional silane precursor is selected from silanes of the formula:

(methacryloyl-O—R¹)—(CH₂)ₙ—Si(R²)ₘ(OR³)₍₃₋ₘ₎
wherein:
n is an integer from 1 to 12;
m is 0, 1, or 2;
R¹ is a divalent alkylene group optionally substituted with one or more C₁-C₆ alkyl groups;
R² is a C₁-C₆ alkyl group or a halogen; and
R³ is independently selected from C₁-C₆ alkyl groups.

In some embodiments, m is 0. In some embodiments, R³ is independently selected from methyl or ethyl. In some embodiments, m is 0 and R³ is independently selected from methyl or ethyl.

In some embodiments, n is 3.

In some embodiments, the methacrylate-functional silane precursor is selected from the group consisting of 3-(trimethoxysilyl)propyl methacrylate, 3-(triethoxysilyl)propyl methacrylate, 3-(methacryloyloxy)propyl methyldimethoxysilane, 3-(methacryloyloxy)propyl methyldiethoxysilane, 3-(methacryloyloxy)propyl dimethylchlorosilane, and 11-(trimethoxysilyl)undecyl methacrylate. In some embodiments, the precursor is a trialkoxysilane selected from 3-(trimethoxysilyl)propyl methacrylate and 3-(triethoxysilyl)propyl methacrylate. In some embodiments the silane precursor is 3-(trimethoxysilyl)propyl methacrylate.

In some embodiments, the disclosure provides compositions comprising an immobilized enzyme, wherein the enzyme is a lipase. In some embodiments, the lipase is Candida antartica lipase B (CalB). In some embodiments, the lipase is selected from Candida antartica lipase B (CalB) and its isoforms.

In some embodiments, the disclosed compositions comprise one or more optional components selected to enhance enzyme performance, improve formulation characteristics, or facilitate manufacturing or use. Such components may be incorporated at one or more stages, including prior to immobilization, during immobilization, following immobilization, or at multiple stages thereof. In some embodiments, the total amount of such components ranges from less than about 0.1 wt % to greater than about 99 wt % of the total composition.

In some embodiments, the disclosure provides compositions comprising an immobilized enzyme, wherein the composition further comprises at least one additive. In some embodiments, at least one additive is selected from salts, sugars, and surfactants. In some embodiments, the salts are selected from calcium chloride, potassium chloride or combinations thereof. In some embodiments, the additive is potassium chloride. In some embodiments, the sugars are selected from glucose, fructose, sucrose, and combinations thereof. In some embodiments, the at least one additive comprises at least one preservative In some embodiments, the additive is a preservative selected from potassium sorbate, sodium benzoate or combinations thereof. In some embodiments, the at least one additive comprises at least one alcohol. In some embodiments the additive alcohol is selected from glycerol, glycol, polyethylene glycols, and polypropylene glycols. In some embodiments, the at least one additive is an ether. In some embodiments, the additive is an ether which is diethylene glycol butyl ether. In some embodiments, the additive comprises combinations of any of the foregoing.

Suitable additives and excipients may include, without limitation, stabilizers; preservatives; surfactants; salts; buffers; ionic modifiers; protective or stress-mitigating agents; activity modifiers; solvents or cosolvents; spacers or conformation-modifying agents; carriers; binders; disintegrants; lubricants; moisture-control agents; pH-adjusting agents; and flow aids. Such components may be organic or inorganic, polymeric or non-polymeric, synthetic or naturally derived.

Non-limiting examples of additives and excipients suitable for use in the compositions of the present disclosure are summarized in Table 1 below, which categorizes representative materials according to their primary function. The categories and examples in Table 1 are intended to illustrate, and not to limit, the scope of the present disclosure, and individual components may serve multiple functions within a given formulation.

TABLE 1
Non-Limiting Exemplary Additives and Excipients

| Component | | |
|---|---|---|
| Category | Primary Role / Function | Representative Examples |
| Stabilizers | Improve thermal, | Polyols (glycerol, sorbitol, mannitol); |
| | chemical, or storage | sugars (trehalose, sucrose, glucose); |
| | stability; reduce | polysaccharides (dextran, pullulan); amino |
| | denaturation or activity | acids (glycine, proline); proteins (gelatin, |
| | loss | bovine serum albumin); polymers |
| | | (polyethylene glycol (PEG), polyvinyl |
| | | alcohol (PVA)) |
| Preservatives / | Inhibit microbial growth | Potassium sorbate; sodium benzoate; |
| Antimicrobials | during storage or use | parabens; phenolic compounds; bronopol; |
| | | isothiazolinones |
| Surfactants / | Improve dispersion, | Non-ionic surfactants (Tween ® 20, |
| Wetting Agents | wettability, substrate | Tween ® 80, Triton ™ X-100); ionic |
| | access, and mass transfer | surfactants (SDS, CTAB); amphoteric |
| | | surfactants; block copolymers (Pluronic ® |
| | | materials) |
| Salts / Ionic | Adjust ionic strength and | Sodium chloride; potassium chloride; |
| Modifiers | enzyme | ammonium sulfate; calcium chloride; |
| | microenvironment | magnesium sulfate; phosphate salts |
| Buffers / pH | Maintain pH during | Phosphate; Tris; HEPES; citrate; acetate; |
| Modifiers | storage or catalytic use | carbonate; borate buffers |
| Protective / | Protect against oxidative, | Antioxidants (ascorbic acid, tocopherols); |
| Stress-Mitigating | thermal, mechanical, | chelating agents (EDTA) |
| Agents | shear, or solvent-induced | |
| | stress | |
| Activity Modifiers | Modulate or tune enzyme | Cofactors or ions (Ca²⁺, Mg²⁺); reversible |
| | activity or selectivity | inhibitors; competitive substrates; affinity |
| | | ligands; substrate analogs |
| Solvents / | Influence substrate | Water; alcohols (ethanol, isopropanol); |
| Cosolvents | solubility or enzyme | ketones; esters; hydrocarbons; ionic |
| | conformation | liquids; deep eutectic solvents, ethers |
| | | (diethylene glycol butyl ether). |
| Anti-caking / Flow | Improve flowability and | Silicon dioxide; calcium silicate; |
| Aids | handling of dry | magnesium stearate; talc |
| | formulations | |
| Conformational | Improve accessibility, | Polyethylene glycol chains; |
| Modifiers | orientation, or | |
| | conformation of | |
| | immobilized enzyme | |
| Carriers / Diluents | Provide bulk, support, or | Silica; alumina; diatomaceous earth; |
| | immobilization substrate | cellulose; starch; polymer beads; resin |
| | | particles |
| Binders / Matrix | Promote cohesion or | Polyvinyl alcohol; alginate; chitosan; |
| Formers | structural integrity | gelatin; polyacrylates |
| Disintegrants / | Facilitate dispersion upon | Sodium starch glycolate; crospovidone; |
| Solubilizers | use | polyethylene glycol |
| Lubricants / | Improve | Magnesium stearate; stearic acid; glyceryl |
| Anti-adhesion | manufacturability and | behenate |
| Agents | processing | |
| Moisture Control | Regulate water content | Humectants (glycerol); desiccants (silica |
| Agents | during storage or use | gel); hygroscopic salts |
| pH-Adjusting | Improve formulation | Acids, bases, or buffering excipients |
| Agents | compatibility and | |
| | stability | |

The specific compounds useful in these roles are not limited to the examples disclosed herein. Any compound now known or later developed that performs a similar function in an immobilized enzyme system may be employed without departing from the scope of the present disclosure.

### Methods:

The disclosure further provides methods for immobilizing an enzyme, the enzyme comprising (a) non-ordered amorphous silica having a silica surface that is functionalized with a methacryl group; and (b) a lipase immobilized on the silica surface that is functionalized with the methacryl group. In some embodiments, the method comprises functionalizing a silica gel using an impregnation technique with a methacryl group precursor; preparing a solution comprising the enzyme stabilized in a buffer; optionally pretreating the functionalized silica; and adding the solution to the functionalized silica and mixing to form an immobilized enzyme.

In some embodiments, the impregnation technique is selected from an incipient wetness impregnation technique and a slurry impregnation technique. In some embodiments, the impregnation technique is an incipient wetness impregnation technique. In some embodiments, the impregnation technique is a slurry impregnation technique.

In some embodiments, the methacryl group precursor is a silane.

In some embodiments, the methacryl group precursor is a methacrylate-functional silane precursor. In some embodiments, the methacrylate-functional silane precursor comprises at least one methacrylate group and at least one hydrolyzable silyl group. In some embodiments, the methacrylate-functional silane precursor is selected from silanes of the formula:

(methacryloyl-O—R¹)—(CH₂)ₙ—Si(R²)ₘ(OR³)₍₃₋ₘ₎
wherein:
n is an integer from 1 to 12;
m is 0, 1, or 2;
R¹ is a divalent alkylene group optionally substituted with one or more C₁-C₆ alkyl groups;
R² is a C₁-C₆ alkyl group or a halogen; and
R³ is independently selected from C₁-C₆ alkyl groups.

In some embodiments, m is 0. In some embodiments, R³ is independently selected from methyl or ethyl. In some embodiments, m is 0 and R³ is independently selected from methyl or ethyl.

In some embodiments, n is 3.

In some embodiments, the methacrylate-functional silane precursor is selected from the group consisting of 3-(trimethoxysilyl)propyl methacrylate, 3-(triethoxysilyl)propyl methacrylate, 3-(methacryloyloxy)propyl methyldimethoxysilane, 3-(methacryloyloxy)propyl methyldiethoxysilane, 3-(methacryloyloxy)propyl dimethylchlorosilane, 6-(trimethoxysilyl)hexyl methacrylate, and 11-(trimethoxysilyl)undecyl methacrylate. In some embodiments, the precursor is a trialkoxysilane selected from 3-(trimethoxysilyl)propyl methacrylate and 3-(triethoxysilyl)propyl methacrylate. In some embodiments the silane precursor is 3-(trimethoxysilyl)propyl methacrylate.

In some embodiments, the methacryl group precursor is used in an amount to achieve an OH:silane molar ratio. In some embodiments, the OH:silane molar ratio is in the range of 10 to 0.5. In some embodiments, the OH:silane molar ratio is in the range of 5 to 1. In some embodiments, the OH:silane molar ratio is in the range of 4 to 1.5. In some embodiments, the OH:silane molar ratio is about 1.5.

In some embodiments, the method comprises functionalizing a silica gel at room temperature.

In some embodiments, the method comprises preparing a solution comprising an enzyme stabilized in a buffer. Suitable buffers include, but are not limited to, acetate, citrate, phosphate, HEPES, PIPES, MES, PES, BIS, TRIS, and MOPS buffers. In certain embodiments, the buffer comprises sodium phosphate. In some embodiments, the buffer has a pH in the range of about 5.0 to about 8.0. In particular embodiments, the pH is approximately 5.0, 5.5, 6.0, 6.5, 7.0, 7.5, or 8.0.

In some embodiments, the method comprises the step of preparing a solution comprising the enzyme stabilized in a buffer, wherein the solution comprising the enzyme stabilized in a buffer has protein concentrations in the range of 0.1 mg/mL to 5 mg/mL. In some embodiments, the solution comprising the enzyme stabilized in a buffer has protein concentrations in the range of 1 mg/mL to 5 mg/mL. In some embodiments, the solution comprising the enzyme stabilized in a buffer has protein concentrations in the range of 0.5 mg/mL to 1.5 mg/mL. In some embodiments, the solution comprising the enzyme stabilized in a buffer has protein concentrations of about 1.0 mg/mL.

In some embodiments, the method comprises pretreating the functionalized silica. In some embodiments, the silica is pretreated with an alcohol. In some embodiments, the alcohol is selected from methanol, ethanol, isopropanol, n-propanol, and butanol. In some embodiments, the silica is pretreated with an ether. In some embodiments, the ether is diethylene glycol butyl ether.

In some embodiments, the method comprises adding an enzyme-buffer solution to functionalized silica and mixing to form an immobilized enzyme. In some embodiments, the solution is added to the functionalized silica in an amount sufficient to achieve a functionalized silica:enzyme-buffer solution weight ratio. In some embodiments, the solution is added to the functionalized silica in an amount sufficient to achieve an functionalized silica:enzyme-buffer solution weight ratio in the range of 1:2 to 1:50. In some embodiments, the solution is added to the functionalized silica in an amount sufficient to achieve an functionalized silica:enzyme-buffer solution weight ratio in the range of 1:10 to 1:30. In some embodiments, the solution is added to the functionalized silica in an amount sufficient to achieve an functionalized silica:enzyme-buffer solution weight ratio in the range of 1:20 to 1:50. In some embodiments, the solution is added to the functionalized silica in an amount sufficient to achieve a functionalized silica:enzyme-buffer solution weight ratio of 1:30. In some embodiments, the solution is added to the functionalized silica in an amount sufficient to achieve an functionalized silica:enzyme-buffer solution weight ratio in the range of 1:10 to 1:20. In some embodiments, the solution is added to the functionalized silica in an amount sufficient to achieve a functionalized silica:enzyme-buffer solution weight ratio of 1:13. In some embodiments, the solution is added to the functionalized silica in an amount sufficient to achieve a functionalized silica:enzyme-buffer solution weight ratio of 1:2, 1:5, 1:10, 1:15, 1:20, 1:25, 1:30, 1:35, 1:40, 1:45, or 1:50.

In some embodiments, once the solution is added to the functionalized silica, mixing occurs at temperatures in the range of 0 °C to 80 °C. In some embodiments, once the solution is added to the functionalized silica, mixing occurs at temperatures in the range of 4 °C to 60 °C. In some embodiments, once the solution is added to the functionalized silica, mixing occurs at temperatures in the range of 10 °C to 40 °C. In some embodiments, once the solution is added to the functionalized silica, mixing occurs at room temperature.

In some embodiments, once the solution is added to the functionalized silica, mixing occurs for at least 30 minutes. In some embodiments, once the solution is added to the functionalized silica, mixing occurs for at least 1 hour. In some embodiments, once the solution is added to the functionalized silica, mixing occurs for at least 2 hours. In some embodiments, once the solution is added to the functionalized silica, mixing occurs for 1-4 hours. In some embodiments, once the solution is added to the functionalized silica, mixing occurs for up to 24 hours.

In some embodiments, the methods described herein further comprise rinsing and drying steps after formation of the immobilized enzyme. In some embodiments, the rinsing step is done with water. In some embodiments, the rinsing step is done with deionized water. In some embodiments, the rinsing step is done with an organic solvent. In some embodiments, the organic solvent is selected from methanol, ethanol, isopropanol, n-propanol, hexanes, cyclohexane, and benzene.

In some embodiments, the drying step occurs above room temperature. In some embodiments, the drying step occurs above 40 °C. In some embodiments, the drying step occurs above 60 °C.

In some embodiments, the method further comprises one or more optional components selected to enhance enzyme performance, improve formulation characteristics, or facilitate manufacturing or use. Such components may be incorporated at one or more stages, including prior to immobilization, during immobilization, following immobilization, or at multiple stages thereof.

In some embodiments, the rinsing step further comprises the use of one or more additives. In some embodiments, at least one additive is selected from salts, sugars, and surfactants. In some embodiments, the salts are selected from calcium chloride, potassium chloride or combinations thereof. In some embodiments, the additive is potassium chloride. In some embodiments, the sugars are selected from glucose, fructose, sucrose, and combinations thereof. In some embodiments, the at least one additive comprises at least one preservative In some embodiments, the additive is a preservative selected from potassium sorbate, sodium benzoate or combinations thereof. In some embodiments, the at least one additive comprises at least one alcohol. In some embodiments the additive alcohol is selected from glycerol, glycol, polyethylene glycols, and polypropylene glycols. In some embodiments, the at least one additive is an ether. In some embodiments, the additive is an ether which is diethylene glycol butyl ether. In some embodiments, the additive comprises combinations of any of the foregoing.

Suitable additives and excipients may include, without limitation, stabilizers; preservatives; surfactants; salts; buffers; ionic modifiers; protective or stress-mitigating agents; activity modifiers; solvents or cosolvents; spacers or conformation-modifying agents; carriers; binders; disintegrants; lubricants; moisture-control agents; pH-adjusting agents; and flow aids. Such components may be organic or inorganic, polymeric or non-polymeric, synthetic or naturally derived.

Non-limiting examples of additives and excipients suitable for use in the compositions of the present disclosure are summarized in Table 1 above, which categorizes representative materials according to their primary function. The categories and examples in Table 1 are intended to illustrate, and not to limit, the scope of the present disclosure, and individual components may serve multiple functions within a given formulation.

The specific compounds useful in these roles are not limited to the examples disclosed herein. Any compound now known or later developed that performs a similar function in an immobilized enzyme system may be employed without departing from the scope of the present disclosure.

### Enzymatic Reactions:

The disclosure further provides methods of performing an enzymatic reaction. In some embodiments, the enzymatic reaction comprises contacting a substrate with an immobilized enzyme comprising: (a) non-ordered amorphous silica having a silica surface that is functionalized with a methacryl group; and (b) a lipase immobilized on the silica surface that is functionalized with the methacryl group; wherein contacting is performed under conditions effective to catalyze conversion of the substrate to a reaction product.

In some embodiments, the enzymatic reaction is a hydrolysis reaction or a transesterification reaction. In some embodiments, the enzymatic reaction is a hydrolysis reaction. In some embodiments, the enzymatic reaction is a transesterification reaction.

In some embodiments, contacting of the substrate with the immobilized enzyme is performed at a temperature in a range of 0 °C to 120 °C. In some embodiments, contacting of the substrate with the immobilized enzyme is performed at a temperature in a range of 50 °C to 100 °C. In some embodiments, contacting of the substrate with the immobilized enzyme is performed at a temperature in a range of 20 °C to 90 °C. In some embodiments, contacting of the substrate with the immobilized enzyme is performed at a temperature in a range of 0 °C to 60 °C. In some embodiments, contacting of the substrate with the immobilized enzyme is performed at a temperature in a range of 0 °C to 40 °C. In some embodiments, contacting of the substrate with the immobilized enzyme is performed at a temperature of about 25 °C.

In some embodiments, contacting of the substrate with the immobilized enzyme is performed at a pH in a range of 5 to 9. In some embodiments, contacting of the substrate with the immobilized enzyme is performed at a pH of about 7.

In some embodiments, the enzymatic reaction further comprises recovering the immobilized enzyme after the contacting and reusing the immobilized enzyme. In some embodiments, the immobilized enzyme is reused in at least 1 additional reaction cycle. In some embodiments, the immobilized enzyme is reused in at least 10 additional reaction cycles. In some embodiments, the immobilized enzyme is reused in at least 1, at least 2, at least 3, at least 4, at least 5, at least 6, at least 7, at least 8, at least 9, or at least 10 additional reaction cycles.

In some embodiments, the enzymatic reaction uses an immobilized enzyme in the form of a composition. In some embodiments, the composition further comprises one or more optional components selected to enhance enzyme performance, improve formulation characteristics, or facilitate manufacturing or use. Such components may be incorporated at one or more stages, including prior to immobilization, during immobilization, following immobilization, or at multiple stages thereof. In some embodiments, the composition comprises an immobilized enzyme and at least one additive. In some embodiments, at least one additive is selected from salts, sugars, and surfactants. In some embodiments, the salts are selected from calcium chloride, potassium chloride or combinations thereof. In some embodiments, the additive is potassium chloride. In some embodiments, the sugars are selected from glucose, fructose, sucrose, and combinations thereof. In some embodiments, the at least one additive comprises at least one preservative In some embodiments, the additive is a preservative selected from potassium sorbate, sodium benzoate or combinations thereof. In some embodiments, the at least one additive comprises at least one alcohol. In some embodiments the additive alcohol is selected from glycerol, glycol, polyethylene glycols, and polypropylene glycols. In some embodiments, the at least one additive is an ether. In some embodiments, the additive is an ether which is diethylene glycol butyl ether. In some embodiments, the additive comprises combinations of any of the foregoing.

Suitable additives and excipients may include, without limitation, stabilizers; preservatives; surfactants; salts; buffers; ionic modifiers; protective or stress-mitigating agents; activity modifiers; solvents or cosolvents; spacers or conformation-modifying agents; carriers; binders; disintegrants; lubricants; moisture-control agents; pH-adjusting agents; and flow aids. Such components may be organic or inorganic, polymeric or non-polymeric, synthetic or naturally derived.

Non-limiting examples of additives and excipients suitable for use in the compositions of the present disclosure are summarized in Table 1 above, which categorizes representative materials according to their primary function. The categories and examples in Table 1 are intended to illustrate, and not to limit, the scope of the present disclosure, and individual components may serve multiple functions within a given formulation.

The specific compounds useful in these roles are not limited to the examples disclosed herein. Any compound now known or later developed that performs a similar function in an immobilized enzyme system may be employed without departing from the scope of the present disclosure.

### EXAMPLES

### Methods of Enzyme Assays:

Immobilization Yield: Immobilization yield (1) is calculated by the following formula:

$$I = \frac{{m}_{1} - {m}_{2}}{{m}_{1}} \times 100 \%$$

in which m₁ is the amount of bio-enzyme (mg) being added during the immobilization process; m₂ is the amount of enzyme (mg) in the supernatant obtained after the preparation of immobilized materials. The concentration of protein was measured using the Sigma-Aldrich Pierce™ Dilution-Free™ Rapid Gold BCA protein assay kit (Catalog #A55861), with bovine serum albumin (BSA) and pure CalB used as protein standards.

NPB Hydrolysis: The enzymatic hydrolytic activity of immobilized CalB product was assessed using p-nitrophenyl butyrate (p-NPB) as the substrate. This assay is based on the hydrolysis of p-NPB by the lipase, which releases p-nitrophenol, a compound that can be quantified spectrophotometrically. The method, described by Femandez-Lafuente et. al., Enzyme and Microbial Technology, 71, 2015, 53-57, herein incorporated by reference in its entirety, was conducted at a temperature of 25 °C, with a reaction time of two minutes. After the reaction, the immobilized CalB was collected by filtration for the next cycle of hydrolysis, to allow for evaluating the potential for recycling and the stability of the derivatives. One enzyme activity unit (PU) was defined as 1 mmol hydrolyzed substrate per minute.

Transesterification of vinyl acetate with n-butanol: The activity of the immobilized CalB for the transesterification reaction was determined by measuring the acetaldehyde produced from the transesterification of vinyl acetate with n-butanol in n-hexane. This was done using the 3-methyl-2-benzothiazolinone hydrazine (MBTH) derivatization method. The MBTH initially reacts with acetaldehyde to produce the aldazine intermediate, which is transformed into a blue tetraaza-pentamethine cyanine (TAPMC) dye via oxidative coupling with a second MBTH molecule. The TAPMC can be detected by the absorbance in 598 nm, and the correlation of absorbance intensity and concentration can be determined using a standard curve of acetaldehyde. One international unit (U) of the transesterification activity was defined as the amount of enzyme that produced 1 mmol of vinyl alcohol per minute.

Propyl Laurate Units (PLU): Propyl Laurate Units (PLU) is a titrimetric method used to quantify lipase activity based on the conversion of lauric acid. The assay is performed in system containing lauric acid and 1-propanol, under 60 °C, during 5 minutes. After incubation, the reaction is quenched and the residual free lauric acid is titrated with standardized NaOH (e.g., 0.1 M) using phenolphthalein or potentiometric detection. The activity is calculated from the difference between blank and sample titration volumes, reflecting the pmol of lauric acid consumed per minute per gram of derivate. 1 PLU is the amount of enzyme activity which generates 1 mol of propyl laurate per minute under described conditions.

Transesterification of 2-phenylethanol with vinyl acetate: The activity of the immobilized lipase preparation was determined in an organic medium using a model transesterification reaction. The immobilized lipase catalyzes the acylation of (R)-1-phenylethanol with vinyl acetate to form (R)-1-phenylethyl acetate and acetaldehyde. The reaction was carried out in methyl tert-butyl ether (MTBE) containing 2 M 1-phenylethanol, 1.2 M vinyl acetate, and 1 vol % dodecane as internal standard. Assays were performed by adding 10 mg of immobilized catalyst per mL of reaction mixture in 1.5 mL tubes, followed by incubation at 35 °C under agitation at 1500 rpm for 30 min to ensure adequate mass transfer. After reaction, the catalyst was allowed to sediment, and a 10 μL aliquot of the supernatant was diluted in 990 μL MTBE for analysis. Product formation was quantified by gas chromatography with flame ionization detection (GC-FID), and enzymatic activity was expressed based on the rate of ester formation under these defined conditions.

In all examples, the CalB enzyme precursor was Lipozyme® CalB-L or Sustine® 111, although other CalB enzyme precursors may also be used and are within the scope of the disclosure.

### Example 1: Silica and Silica Functionalization

Silica gel with a surface area of about 110 m²/g, a pore volume of about 1.0 cc/g, an average pore diameter of about 35 nm, a hydroxyl group concentration of 3 OH/nm², and a particle size of 100-300 μm was functionalized with a methacryl group using the incipient wetness impregnation technique using 3-(trimethoxysilyl)propyl methacrylate (MPTS) as the methacryl group precursor. 9 g of MPTS was dissolved in methanol and the solution was impregnated on to the silica via incipient wetness impregnation technique. The amount of silane corresponds to an OH:silane ratio of 1.5. The impregnated material was allowed to react for at least 30 minutes at room temperature, and then the solvent optionally evaporated under heat and vacuum.

After the designated reaction time and before solvent evaporation, the material was optionally washed using water or an organic solvent, such as methanol or hexane, to wash away any of the silane precursor that is not covalently bonded to the silica.

The dried product was optionally washed using water or an organic solvent, like methanol or hexane, to wash away any of the silane precursor that is not covalently bonded to the silica, and the product was then optionally re-dried before use in enzyme immobilization.

### Example 2: Enzyme Immobilization and Performance

The enzyme was immobilized on the functionalized silica support from Example 1 by adsorption. A CalB precursor solution was stabilized in a sodium phosphate buffer (~25 mM, pH 7.0) to give the enzyme-buffer solution. Protein concentrations ranging from 0.5 mg/mL to 1.5 mg/mL were targeted for the enzyme-buffer solution. Prior to immobilization, the functionalized silica was pretreated with 5% isopropanol for 10 minutes at room temperature to enhance its dispersion in the enzyme-buffer solution. Subsequently, the enzyme-buffer solution was added to the functionalized silica in the functionalized silica:enzyme-buffer solution weight ratio of 1:30 respectively and gently mixed at room temperature for a duration of one to four hours. The pH and protein concentration were monitored to assess the efficacy of immobilization. Following immobilization, the derivative was thoroughly rinsed with deionized water. Finally, the product was dried in a mechanical convection incubator set at 27 °C until the derivate was dry. The immobilization yield for the immobilized CalB ranged from 70% to 85% when using a 1.5 mg/mL protein in the enzyme-buffer solution.

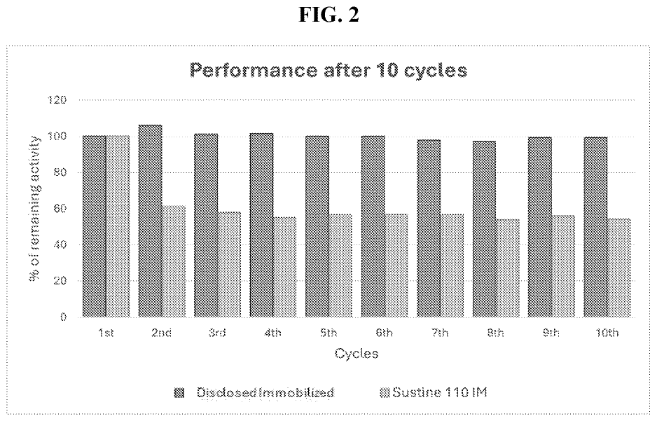

The hydrolysis activity of the immobilized enzyme product was measured using the p-NPB hydrolysis method. Under the tested conditions, the hydrolytic activity of the CalB immobilized on silica was comparable to that of Sustine® 110 IM, with both showing an activity of 34,000 U per gram of product during the first cycle of use. A stability test was conducted under the same conditions to assess the performance of the derivatives over ten reaction cycles. The CalB immobilized on silica retained 100% of its initial activity, while the Sustine 110 IM experienced a 40% decrease in activity after the second cycle. These results are shown in FIG. 2.

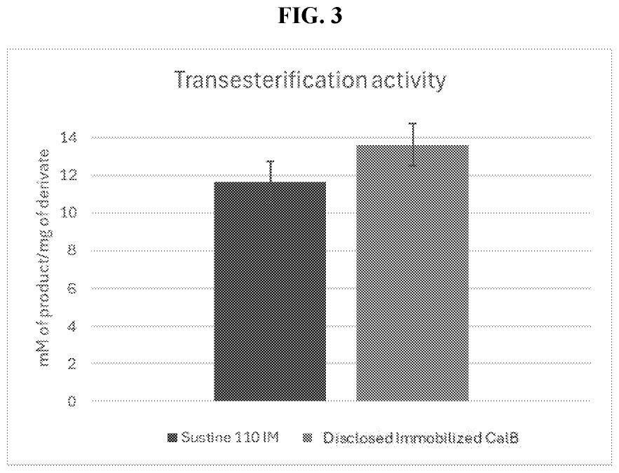

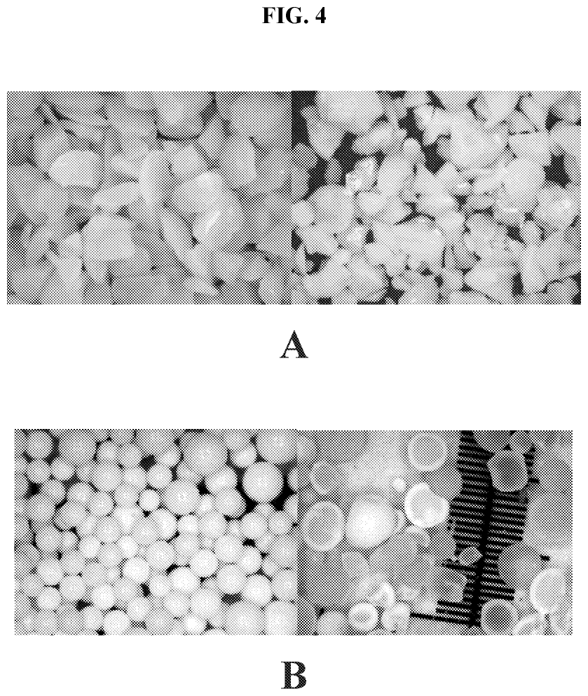

FIG. 3 shows the activity for transesterification of vinyl acetate with n-butanol for the disclosed CalB immobilized on silica is similar to Sustine® 110 IM.

### Example 3: Influence of Pore Diameter and Enzyme Amount

In certain conditions, mesoporous silica allows the enzyme to disperse on the large internal surfaces and pores, which in turn improves the activity of the catalyst system is a significant platform for enzyme immobilization. The modulation of size and diameter of pores can be a dominant factor due to the surface area modulation and transport phenomena.

CalB immobilization was performed on methacryl functionalized silica supports with pore diameters between 10 nm and 80 nm under identical conditions. These silicas had pore volumes of about 1.0 cc/g and particle size 300-700 micron. The immobilization was carried out in a 20 mM sodium phosphate buffer with IPA (5 mL/g-silica), with varying amounts of enzyme precursor respectively. The solution was mixed for four hours under mild agitation at 25 °C. Following immobilization, the derivative was thoroughly washed thrice using 5 mL of sodium phosphate buffer (~20 mM, pH 7.0) per gram of support. Finally, the product was dried in a vacuum oven at 45 °C overnight until the moisture content reached between 0.5% to 2.0% by weight.

For the 10 nm pore diameter silica, no immobilization yield or catalytic activity was observed for the enzyme amounts tested.

For the 17 nm pore diameter silica, an immobilization yield of 81.6% was achieved at low enzyme amounts (2 mL enzyme precursor/g-functionalized silica) using 3 wt % functionalized silica in solution, with measured hydrolytic activity via the p-NPB method of 38,000 PU.

For the 37 nm pore diameter silica, the immobilization yields between ~6.5% and 56.5% were obtained at moderate enzyme amounts (about 5.5 ml enzyme precursor/g-functionalized silica), 1 wt % and 7 wt % silica in solution respectively, accompanied by strong MBTH activity (up to 122.6 U) and high p-NPB values (40,000-65,000 PU). This pore size supports both effective immobilization and good catalytic performance.

For the 50 nm pore diameter silica using 3 wt % functionalized silica in solution, the highest immobilization yield (100%) is achieved at low enzyme amounts (2 ml enzyme precursor/g-functionalized silica) with good catalytic activity (MBTH 82.61 U; p-NPB ~44,957 PU). At higher enzyme amounts (9 ml enzyme precursor/g-functionalized silica), the MBTH activity increases up to 126.6 U.

For the 80 nm pore diameter silica, moderate immobilization yield (55.1%) is observed at 5.5 ml enzyme precuresor/g-functionalized silica, 7 wt % functionalized silica in solution, with MBTH activity of 60.51 U and hydrolytic activity of p-NPB ~69,223 PU)

### Example 4: Influence of Temperature and Time on Immobilization Yield

Contact time during the immobilization step is a critical parameter in the development of immobilized enzyme systems, as it directly influences enzyme loading, distribution within the support, and preservation of catalytic activity. Immobilization is a time-dependent process governed by adsorption kinetics, diffusion into the porous matrix, and the establishment of enzyme-support interactions. While longer contact times can promote increased enzyme uptake, they may also lead to overcrowding, diffusional limitations, or unfavorable conformational changes that negatively impact activity. Therefore, optimizing immobilization time is essential to achieve a balance between maximizing enzyme loading and maintaining high catalytic efficiency.

Lipase immobilization was evaluated over varying contact times ranging from 1 to 8 hours. A CalB precursor solution was stabilized in a sodium phosphate buffer (~20 mM, pH 7.0) to give the enzyme-buffer solution. The enzyme:buffer solution ratio was 1:1.25 Diethylene glycol butyl ether (0.025 ml/g-functionalized silica) was added to enzyme-buffer solution prior to the immobilization. Subsequently, the enzyme-buffer solution was added to the functionalized silica (silica avg. pore diameter ~55 nm, particle size of 300-700 microns) in the functionalized silica:enzyme-buffer solution weight ratio 1:13 respectively and gently mixed for various combinations of contact time and temperature respectively. Following immobilization, the derivative was thoroughly washed thrice using 5 mL of sodium phosphate buffer (~20 mM, pH 7.0) per gram of support. Finally, the product was dried in a vacuum oven at 45 °C overnight.

Aliquots of the enzyme solution (supernatant) were taken every hour, and their enzyme concentration were determined using BCA-Gold methodology with BSA as standards. The enzyme concentration in the supernatant was compared with the initial solution (pre-immobilization) to determine the Immobilization Yield.

Immobilization yield increased with time until 3 hours and the extended immobilization times didn't result in significant increment on immobilization yield or activity. Optimal performance was observed at immobilization times between one to four hours, balancing enzyme loading and activity.

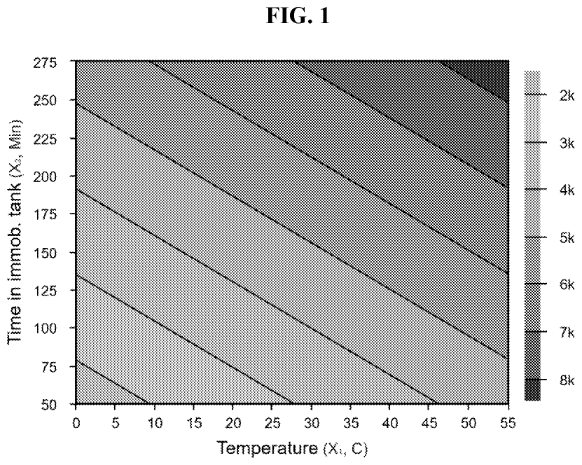

Conventional immobilization procedures are typically conducted at room temperature or under refrigerated conditions due to the thermal sensitivity of enzymes. In the present study, it was confirmed that both temperature and time exerted a positive influence on enzyme uptake and, consequently, on the activity of the derivatives (results shown in FIG. 1). The highest immobilization yield was achieved at temperatures above 35 °C and with immobilization times exceeding two hours under agitation.

### Example 5: Influence of Salt Additives

In biological formulations, additives are widely used to stabilize proteins, modulate solubility, control ionic strength, and preserve functional conformation during processing and storage. Salts, for example, can influence electrostatic interactions and protein folding, The effect of various salt additives on enzyme immobilization was investigated to better understand how the chemical environment influences enzyme-support interactions, loading efficiency, and catalytic performance.

The immobilization process was carried out using a CalB precursor solution stabilized in a sodium phosphate buffer (~20 mM, pH 7.0) to give the enzyme-buffer solution. The enzyme:buffer solution ratio was 1:1.25. The IPA (0.25 ml/g-functionalized silica) was added to enzyme-buffer solution prior to the immobilization. Subsequently, the enzyme-buffer solution was added to the functionalized silica (silica avg. pore diameter ~55 nm, particle size of 300-700 microns) in the functionalized silica:enzyme-buffer solution weight ratio 1:13 respectively and gently mixed at room temperature (about 25 C) for a duration for around four hours in the presence of various salts (NaCl, KCl, CoCl₂) with concentrations ranging from 25 mM to 50 mM. Compared to the control (no additive), monovalent salts slightly reduced immobilization yield but maintained or improved the catalyst activity as measure by the PLU method.

Among the various ions evaluated, potassium chloride, at concentrations ranging from 20 mM to 70 mM, demonstrated the most favorable results in terms of activity enhancement with an increase in enzymatic activity compared to control of approximately 30-50% as measured by PLU and 10-30% for the transesterification of 2-phenylethanol with vinyl acetate. In contrast, CoCl₂ led to reduced activity, indicating that it inhibits enzyme action. These findings indicate that salt additives can influence enzyme immobilization and activity.

| | | | | Conversion (%) |
|---|---|---|---|---|
| | Salt | | | Transesterification |
| | Concen- | Immobilization | | of 2-phenylethanol |
| Salt | tration | Yield (%) | PLU/g | with vinyl acetate |
| (Control) | — | 33.7 | 2029 | 8.2 |
| KCl | 25 mM | 30.8 | 3070 | 9.3 |
| KCl | 50 mM | 29.9 | 2751 | 11.0 |
| NaCl | 25 mM | 29.4 | 2235 | 9.0 |
| NaCl | 50 mM | 29.6 | 2225 | 10.0 |
| CoCl₂ | 25 mM | 28.7 | 2232 | 7.7 |
| CoCl₂ | 50 mM | 28.2 | 2476 | 6.8 |

### Example 6: Ether as an Additive

A CalB precursor solution was stabilized in a sodium phosphate buffer (~20 mM, pH 7.0) to give the enzyme-buffer solution. The enzyme:buffer solution ratio was 1:1.25. Diethylene glycol butyl ether (0.025 ml/g-functionalized silica) was added to enzyme-buffer solution prior to the immobilization. Subsequently, the enzyme-buffer solution was added to the functionalized silica (silica avg. pore diameter ~55 nm, particle size of 300-700 microns) in the functionalized silica:enzyme-buffer solution weight ratio 1:13 respectively and gently mixed at 50 °C for a duration for around four hours. Following immobilization, the derivative was thoroughly washed thrice using 5 mL of sodium phosphate buffer (~20 mM, pH 7.0) per gram of support. Finally, the product was dried in a vacuum oven at 45 °C overnight. The immobilization yield was 80%, and activity was 6500 PLU/g.

### Example 7: PEG as an Additive

A CalB precursor solution was stabilized in a sodium phosphate buffer (~20 mM, pH 7.0) to give the enzyme-buffer solution. The enzyme:buffer solution ratio was 1:1.25 Diethylene glycol butyl ether (0.025 ml/g-functionalized silica) was added to enzyme-buffer solution prior to the immobilization. Subsequently, the enzyme-buffer solution was added to the functionalized silica (silica avg. pore diameter ~55 nm, particle size of 300-700 microns) in the functionalized silica:enzyme-buffer solution weight ratio 1:13 respectively and gently mixed at 50 °C for a duration for around four hours. Following immobilization, the derivative was thoroughly washed thrice using 5 ml per gram of silica with a 3% solution of PEG 400. Finally, the product was dried in a vacuum oven at 45 °C overnight. The immobilization yield was 65%, and activity was 5700 PLU/g.

### Example 8: Extended Immobilization Contact Time

A CalB precursor solution was stabilized in a sodium phosphate buffer (~20 mM, pH 7.0) to give the enzyme-buffer solution. The enzyme:buffer solution ratio was 1:1.25:Diethylene glycol butyl ether (0.025 ml/g-functionalized silica) was added to enzyme-buffer solution prior to the immobilization. Subsequently, the enzyme-buffer solution was added to the functionalized silica (silica avg. pore diameter ~55 nm, particle size of 300-700 microns) in the functionalized silica:enzyme-buffer solution weight ratio 1:13 respectively and gently mixed at 10 °C for a duration for around 16 hours. Following immobilization, the derivative was thoroughly washed thrice using 5 mL of sodium phosphate buffer (~20 mM, pH 7.0) per gram of support. Finally, the product was dried in a vacuum oven at 45 °C overnight. The immobilization yield was 75%, and activity was 3900 PLU/g.

### Example 9: Surfactant as an Additive

A CalB precursor solution was stabilized in a sodium phosphate buffer (~20 mM, pH 7.0) to give the enzyme-buffer solution. The enzyme:buffer solution ratio was 1:1.25 The surfactant, Triton-X (12 uL/g-functionalized silica), was added to enzyme-buffer solution prior to the immobilization. Subsequently, the enzyme-buffer solution was added to the functionalized silica (silica avg. pore diameter ~55 nm, particle size of 300-700 microns) in the functionalized silica:enzyme-buffer solution weight ratio 1:13 respectively and gently mixed at 50 °C for a duration for around four hours. Following immobilization, the derivative was thoroughly washed thrice using 5 mL of sodium phosphate buffer (~20 mM, pH 7.0) per gram of support. Finally, the product was dried in a vacuum oven at 45 °C overnight. The immobilization yield was 70%, and activity was 3300 PLU/g.

### Example 10: Cetyl Palmitate Synthesis

The immobilized enzyme on functionalized silica product from Example 6 was evaluated as a heterogeneous esterification catalyst for the synthesis of cetyl palmitate, a high-value wax ester widely commercialized in the personal care and lubricant industries. Cetyl palmitate is obtained through the direct esterification of palmitic acid with cetyl alcohol.

Initial catalytic assays were conducted at 70 °C, a temperature considered relevant for industrial applications, in a 2-liter stirred tank reactor. The catalytic performance observed under these conditions was comparable to that of the benchmark catalyst (Sustine® 1101M). CalB immobilized on silica demonstrated superior mechanical resistance compared to Sustine® 110IM, which employs polymeric resins for immobilizing CalB. For example, FIG. 5A shows the CalB immobilized on silica before and after 2 cycles of use at a reaction temperature of 85 °C. FIG. 5B shows Sustine® 110IM before and after 3 cycles of use at a reaction temperature of 70 °C.

Additionally, CalB immobilized on silica exhibited a higher settling velocity and, consequently, improved solid-liquid separation behavior relative to Sustine® 110IM, representing a distinct operational advantage.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Other embodiments of the compositions and methods of the present disclosure will be apparent to those skilled in the art from consideration of the specification and practice of the embodiments disclosed herein. It is intended that the specification and examples be considered as exemplary only, with the true scope of this application being indicated by the following claims.

The foregoing description is presented for purposes of illustration. It is not exhaustive and is not limited to precise forms or embodiments disclosed. Modifications and adaptations of the embodiments will be apparent from consideration of the specification and practice of the disclosed embodiments. While certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of this disclosure are apparent from this detailed specification, and thus, it is intended that the appended claims cover all compositions, compositions, and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Throughout this application, various embodiments of the present disclosure may be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numeric values within that range. For example, description of a range such as from 1 to 6 should be considered to include subranges such as from 1 to 3, from 1 to 4, from 1 to 5, from 2 to 4, from 2 to 6, from 3 to 6, and so forth, as well as individual numbers within that range, for example, 1, 2, 3, 4, 5, and 6. This applies regardless of the breadth of the range.

Other embodiments will be apparent from consideration of the specification and practice of the embodiments disclosed herein. It is intended that the specification and examples be considered as example only, with a true scope and spirit of the disclosed embodiments being indicated by the following claims.

Examples of inventive concepts are contained in the following clauses which are an integral part of this disclosure.

Clause 1. A composition comprising an immobilized enzyme, wherein the immobilized enzyme comprises: non-ordered amorphous silica having a silica surface that is functionalized with a methacryl group; and a lipase immobilized on the silica surface that is functionalized with the methacryl group.

Clause 2. The composition of clause 1, wherein the immobilized enzyme comprises amorphous silica having a particle size ranging from 10 μm to 10 mm.

Clause 3. The composition of clause 1 or 2, wherein the immobilized enzyme comprises amorphous silica having an average particle size from 100 μm to 300 μm, inclusive.

Clause 4. The composition of any one of clauses 1 to 3, wherein the immobilized enzyme comprises amorphous silica having an average particle size from 300 μm to 700 μm, inclusive.

Clause 5. The composition of clauses 1 to 4, wherein the immobilized enzyme comprises amorphous silica having an average pore diameter ranging from 15-150 nm.

Clause 6. The composition of clauses 1 to 5, wherein the immobilized enzyme comprises amorphous silica having an average pore diameter in the range of 20 nm to 100 nm.

Clause 7. The composition of clauses 1 to 6, wherein the immobilized enzyme comprises amorphous silica having an average pore diameter in the range of 30 nm to 80 nm.

Clause 8. The composition of clauses 1 to 7, wherein the immobilized enzyme comprises amorphous silica having an average pore diameter in the range of 40 nm to 60 nm.

Clause 9. The composition of clauses 1 to 8, wherein the immobilized enzyme comprises amorphous silica having a surface area in the range of 10 m²/g to 500 m²/g.

Clause 10. The composition of clauses 1 to 9, wherein the immobilized enzyme comprises amorphous silica having a surface area ranging from 30 m²/g to 150 m²/g.

Clause 11. The composition of clauses 1 to 10, wherein the immobilized enzyme comprises amorphous silica having a surface area ranging from 60 m²/g to 120 m²/g.

Clause 12. The composition of clauses 1 to 11, wherein the immobilized enzyme comprises amorphous silica having a pore volume ranging from 0.1 cc/g to 3.5 cc/g.

Clause 13. The composition of clauses 1 to 12, wherein the immobilized enzyme comprises amorphous silica having a pore volume ranging from 0.5 cc/g to 2.0 cc/g.

Clause 14. The composition of clauses 1 to 13, wherein the immobilized enzyme comprises amorphous silica having a pore volume ranging from 0.8 cc/g to 1.2 cc/g.

Clause 15. The composition of clauses 1 to 14, wherein the immobilized enzyme comprises amorphous silica having a hydroxyl group concentration ranging from 0.1 OH/nm2-5 OH/nm2.

Clause 16. The composition of clauses 1 to 15, wherein the immobilized enzyme comprises amorphous silica having a hydroxyl group concentration ranging from 1 OH/nm2-5 OH/nm2.

Clause 17. The composition of clauses 1 to 16, wherein the lipase Candida antartica lipase B (CalB) and their isoforms.

Clause 18. The composition of clauses 1 to 17, further comprising at least one additive.

Clause 19. The composition of clauses 1 to 18, wherein the at least one additive is selected from salts, sugars, surfactants, preservatives, alcohols and ethers.

Clause 20. The composition of clauses 1 to 19, wherein the salts comprise calcium chloride, potassium chloride or combinations thereof.

Clause 21. The composition of clauses 1 to 20, wherein the sugars comprise glucose, fructose, sucrose, and combinations thereof.

Clause 22. The composition of clauses 1 to 21, wherein the preservative comprises potassium sorbate, sodium benzoate and combinations thereof.

Clause 23. The composition of clauses 1 to 22, wherein the alcohol is selected from glycerol, glycol, polyethylene glycols, and polypropylene glycols.

Clause 24. The composition of clauses 1 to 23, wherein the ether is diethylene glycol butyl ether.

Clause 25. A method for immobilizing an enzyme, the immobilized enzyme comprising: non-ordered amorphous silica having a silica surface that is functionalized with a methacryl group; and a lipase immobilized on the silica surface that is functionalized with the methacryl group, the method comprising: functionalizing a silica gel using an impregnation technique with a methacryl group precursor; preparing a solution comprising the enzyme stabilized in a buffer; optionally pretreating the functionalized silica; and adding the solution to the functionalized silica and mixing to form an immobilized enzyme.

Clause 26. The method of clause 25, wherein the impregnation technique is selected from an incipient wetness impregnation technique and a slurry impregnation technique.

Clause 27. The method of clauses 25 to 26, wherein the methacryl group precursor comprises at least one methacrylate group and at least one hydrolyzable silyl group, wherein the precursor is selected from silanes of the formula:

(methacryloyl-O—R¹)—(CH₂)ₙ—Si(R²)ₘ(OR³)₍₃₋ₘ₎
wherein:
n is an integer from 1 to 12;
m is 0, 1, or 2;
R¹ is a divalent alkylene group optionally substituted with one or more C₁-C₆ alkyl groups;
R² is a C₁-C₆ alkyl group or a halogen; and
R³ is independently selected from C₁-C₆ alkyl groups.

Clause 28. The method of clause 27, wherein m is 0 and R³ is independently selected from methyl or ethyl.

Clause 29. The method of clause 27, wherein n is 3.

Clause 30. The method of clause 25 wherein the methacryl group precursor is selected from the group comprising:
(a) 3-(trimethoxysilyl)propyl methacrylate;
(b) 3-(triethoxysilyl)propyl methacrylate;
(c) 3-(methacryloyloxy)propyl methyldimethoxysilane;
(d) 3-(methacryloyloxy)propyl methyldiethoxysilane;
(e) 3-(methacryloyloxy)propyl dimethylchlorosilane;
(f) 6-(trimethoxysilyl)hexyl methacrylate; and
(g) 11-(trimethoxysilyl)undecyl methacrylate.

Clause 31. The method of clause 25, wherein the precursor is a trialkoxysilane selected from 3-(trimethoxysilyl)propyl methacrylate or 3-(triethoxysilyl)propyl methacrylate.

Clause 32. The method of clause 25 wherein the silane precursor is 3-(trimethoxysilyl)propyl methacrylate

Clause 33. The method of clauses 25 to 32, wherein silane used is in an amount to achieve an OH:silane molar ratio of 10 to 0.5.

Clause 34. The method of clauses 25 to 33, wherein silane used is in an amount to achieve an OH:silane molar ratio of 4 to 1.5.

Clause 35. The method of clauses 25 to 34, wherein functionalizing the silica gel is done at room temperature.

Clause 36. The method of clauses 25 to 35, wherein the buffer comprises sodium phosphate.

Clause 37. The method of clauses 25 to 36, wherein the solution comprising the enzyme stabilized in a buffer has protein concentrations in the range of 0.5 mg/mL to 1.5 mg/mL.

Clause 38. The method of clauses 25 to 37, wherein the solution comprising the enzyme stabilized in a buffer has protein concentrations in the range of 1 mg/mL to 5 mg/mL.

Clause 39. The method of clauses 25 to 38, wherein the silica is pretreated with an alcohol.

Clause 40. The method of clauses 25 to 39, wherein the alcohol is selected from methanol, ethanol, isopropanol, and n-propanol.

Clause 41. The method of clauses 25 to 40, wherein the silica is pretreated with an ether.

Clause 42. The method of clauses 25 to 41, wherein the ether is diethylene glycol butyl ether.

Clause 43. The method of clauses 25 to 42, wherein the solution is added to the functionalized silica in an amount sufficient to achieve a functionalized silica:enzyme buffer weight ratio in the range of 1:2 to 1:50.

Clause 44. The method of clauses 25 to 43, wherein the solution is added to the functionalized silica in an amount sufficient to achieve a functionalized silica:enzyme buffer weight ratio in the range of 1:10 to 1:30.

Clause 45. The method of clauses 25 to 44, wherein the solution is added to the functionalized silica in an amount sufficient to achieve a functionalized silica:enzyme buffer weight ratio of 1:13.

Clause 46. The method of clauses 25 to 45, wherein the solution is added to the functionalized silica in an amount sufficient to achieve functionalized silica:enzyme buffer weight ratio of 1:30.

Clause 47. The method of clauses 25 to 46, wherein once the solution is added to the functionalized silica mixing occurs at temperatures in the range of 4 °C to 60 °C for at least 1 hour.

Clause 48. The method of clauses 25 to 47, further comprising rinsing and drying steps after formation of the immobilized enzyme, wherein the rinsing step is done with deionized water and the drying in step occurs above room temperature.

Clause 49. The method of clauses 25 to 48, wherein the rinsing step further comprises the use of one or more additives.

Clause 50. The method of clauses 25 to 49, wherein the at least one additive is selected from salts, sugars, surfactants, preservatives, alcohols and ethers.

Clause 51. The method of clauses 25 to 50, wherein the salts comprise calcium chloride, potassium chloride or combinations thereof.

Clause 52. The method of clauses 25 to 51, wherein the sugars comprise glucose, fructose, sucrose, and combinations thereof.

Clause 53. The method of clauses 25 to 52, wherein the preservative comprises potassium sorbate, sodium benzoate and combinations thereof.

Clause 54. The method of clauses 25 to 53, wherein the alcohol is selected from glycerol, glycol, polyethylene glycols, and polypropylene glycols.

Clause 55. The method of clauses 25 to 54, wherein the ether is diethylene glycol butyl ether.

Clause 56. A method of performing an enzymatic reaction, the method comprising: contacting a substrate with an immobilized enzyme comprising: non-ordered amorphous silica having a silica surface that is functionalized with a methacryl group; and a lipase immobilized on the silica surface that is functionalized with the methacryl group, wherein contacting is performed under conditions effective to catalyze conversion of the substrate to a reaction product.

Clause 57. The method of clause 56, wherein the enzymatic reaction is a hydrolysis reaction or a transesterification reaction.

Clause 58. The method of clause 56 or 57, wherein contacting is performed at a temperature in a range of 0 °C to 120 °C and at a pH in a range of 5 to 9.

Clause 59. The method of clauses 56 to 58, wherein contacting is performed at a temperature in a range of 20 °C to 90 °C.

Clause 60. The method of clauses 56 to 59, further comprising recovering the immobilized enzyme after the contacting and reusing the immobilized enzyme in at least 1 additional reaction cycles.

Clause 61. The method of clauses 56 to 60, further comprising recovering the immobilized enzyme after the contacting and reusing the immobilized enzyme in at least 10 additional reaction cycles.

Clause 62. The method of clauses 56 to 61, wherein the immobilized enzyme is in the form of a composition that further includes at least one additive selected from salts or sugars, at least one preservative, or combinations thereof.

Disclosed embodiments may include any one of the following bullet-pointed features alone or in combination with one or more other bullet-pointed features, whether implemented in a composition, composition, and/or methods described herein.
an immobilized enzyme.
an immobilized enzyme comprising non-ordered amorphous silica having a silica surface that is functionalized with a methacryl group.
an immobilized enzyme comprising a lipase immobilized on the silica surface that is functionalized with the methacryl group.
an immobilized enzyme comprises amorphous silica having a particle size ranging from 10 μm to 10 mm.
an immobilized enzyme comprises amorphous silica having an average particle size from 100 μm to 300 μm, inclusive.
an immobilized enzyme comprises amorphous silica having an average particle size from 300 μm to 700 μm, inclusive.
an immobilized enzyme comprises amorphous silica having an average pore diameter ranging from 10-150 nm.
an immobilized enzyme comprises amorphous silica having an average pore diameter in the range of 20 nm to 100 nm.
an immobilized enzyme comprises amorphous silica having an average pore diameter in the range of 30 nm to 80 nm.
an immobilized enzyme comprises amorphous silica having an average pore diameter in the range of 40 nm to 60 nm.
an immobilized enzyme comprises amorphous silica having a surface area in the range of 10 m2/g to 500 m2/g.
an immobilized enzyme comprises amorphous silica having a surface area ranging from 30 m2/g to 150 m2/g.
an immobilized enzyme comprises amorphous silica having a surface area ranging from 60 m2/g to 120 m2/g.
an immobilized enzyme comprises amorphous silica having a pore volume ranging from 0.1 cc/g to 3.5 cc/g.
an immobilized enzyme comprises amorphous silica having a pore volume ranging from 0.5 cc/g to 2.0 cc/g.
an immobilized enzyme comprises amorphous silica having a pore volume ranging from 0.8 cc/g to 1.2 cc/g.
an immobilized enzyme comprises amorphous silica having a hydroxyl group concentration ranging from 0.1 OH/nm2-5 OH/nm2.
an immobilized enzyme comprises amorphous silica having a hydroxyl group concentration ranging from 1 OH/nm2-5 OH/nm2.
a lipase Candida antartica lipase B (CalB) and their isoforms.
an immobilized enzyme further comprising at least one additive.
at least one additive is selected from salts, sugars, surfactants, preservatives, alcohols and ethers.
at least one additive in which salts comprise calcium chloride, potassium chloride or combinations thereof.
at least one additive in which sugars comprise glucose, fructose, sucrose, and combinations thereof.
at least one additive in which preservatives comprise potassium sorbate, sodium benzoate and combinations thereof.
at least one preservative in which the alcohol is selected from glycerol, glycol, polyethylene glycols, and polypropylene glycols.
ether which is diethylene glycol butyl ether.
a method for immobilizing an enzyme as described herein.
a method comprising functionalizing a silica gel using an impregnation technique with a methacryl group precursor.
a method comprising preparing a solution comprising the enzyme stabilized in a buffer
a method comprising pretreating the functionalized silica; and adding the solution to the functionalized silica and mixing to form an immobilized enzyme.
a method comprising an impregnation technique is selected from an incipient wetness impregnation technique and a slurry impregnation technique.
a method wherein the methacryl group precursor comprises at least one methacrylate group and at least one hydrolyzable silyl group, wherein the precursor is selected from silanes of the formula:

(methacryloyl-O—R¹)—(CH₂)ₙ—Si(R²)ₘ(OR³)₍₃₋ₘ₎
wherein:
n is an integer from 1 to 12;
m is 0, 1, or 2;
R¹ is a divalent alkylene group optionally substituted with one or more C₁-C₆ alkyl groups;
R² is a C₁-C₆ alkyl group or a halogen; and
R³ is independently selected from C₁-C₆ alkyl groups.
a method wherein m is 0 and R³ is independently selected from methyl or ethyl.
a method wherein n is 3.
a method wherein the methacryl group precursor is selected from the group comprising:
(a) 3-(trimethoxysilyl)propyl methacrylate;
(b) 3-(triethoxysilyl)propyl methacrylate;
(c) 3-(methacryloyloxy)propyl methyldimethoxysilane;
(d) 3-(methacryloyloxy)propyl methyldiethoxysilane;
(e) 3-(methacryloyloxy)propyl dimethylchlorosilane;
(f) 6-(trimethoxysilyl)hexyl methacrylate; and
(g) 11-(trimethoxysilyl)undecyl methacrylate.
a method wherein the precursor is a trialkoxysilane selected from 3-(trimethoxysilyl)propyl methacrylate or 3-(triethoxysilyl)propyl methacrylate.
a method wherein the precursor is 3-(trimethoxysilyl)propyl methacrylate
a method comprising a silane used is in an amount to achieve an OH:silane molar ratio of 10 to 0.5.
a method comprising a silane used is in an amount to achieve an OH:silane molar ratio of 4 to 1.5.
a method comprising wherein functionalizing the silica gel is done at room temperature.
a method wherein the buffer comprises sodium phosphate.
a method comprising wherein the solution comprising the enzyme stabilized in a buffer has protein concentrations in the range of 0.5 mg/mL to 1.5 mg/mL.
a method comprising, wherein the solution comprising the enzyme stabilized in a buffer has protein concentrations in the range of 1 mg/mL to 5 mg/mL.
a method comprising, wherein the silica is pretreated with an alcohol.
a method comprising, wherein the alcohol is selected from methanol, ethanol, isopropanol, and n-propanol.
a method comprising, wherein the silica is pretreated with an ether.
a method comprising, wherein the ether is diethylene glycol butyl ether.
a method comprising, wherein the solution is added to the functionalized silica in an amount sufficient to achieve a functionalized silica:enzyme-buffer solution:weight ratio of in the range of 1:2 to 1:50.
a method comprising, wherein the solution is added to the functionalized silica in an amount sufficient to achieve a functionalized silica:enzyme-buffer solution:weight ratio of in the range of 1:10 to 1:30.
a method comprising, wherein the solution is added to the functionalized silica in an amount sufficient to achieve a functionalized silica:enzyme-buffer solution weight ratio of 1:13.
a method comprising, wherein the solution is added to the functionalized silica in an amount sufficient to achieve a functionalized silica:enzyme-buffer solution weight ratio of in the range of 1:30.
a method comprising, wherein once the solution is added to the functionalized silica mixing occurs at temperatures in the range of 4 °C to 60 °C for at least 1 hour.
a method comprising, further comprising rinsing and drying steps after formation of the immobilized enzyme, wherein the rinsing step is done with deionized water and the drying in step occurs above room temperature.
a method comprising, wherein the rinsing step further comprises the use of one or more additives.
a method comprising, wherein the at least one additive is selected from salts, sugars, surfactants, preservatives, alcohols and ethers.
a method comprising, wherein the salts comprise calcium chloride, potassium chloride or combinations thereof.
a method comprising, wherein the sugars comprise glucose, fructose, sucrose, and combinations thereof.
a method comprising, wherein the at least one additive comprises at least one preservative which comprises potassium sorbate, sodium benzoate and combinations thereof
a method comprising, wherein the alcohol is selected from glycerol, glycol, polyethylene glycols, and polypropylene glycols.
a method comprising, wherein the ether is diethylene glycol butyl ether.
a method of performing an enzymatic reaction.
a method comprising: contacting a substrate with an immobilized enzyme comprising: non-ordered amorphous silica having a silica surface that is functionalized with a methacryl group; and a lipase immobilized on the silica surface that is functionalized with the methacryl group, wherein contacting is performed under conditions effective to catalyze conversion of the substrate to a reaction product.
a method wherein the enzymatic reaction is a hydrolysis reaction or a transesterification reaction.
a method wherein contacting is performed at a temperature in a range of 0 °C to 120 °C and at a pH in a range of 5 to 9.
a method wherein contacting is performed at a temperature in a range of 20 °C to 90 °C.
a method comprising recovering the immobilized enzyme after the contacting and reusing the immobilized enzyme in at least 1 additional reaction cycles.
a method comprising recovering the immobilized enzyme after the contacting and reusing the immobilized enzyme in at least 10 additional reaction cycles.
a method wherein the immobilized enzyme is in the form of a composition that further includes at least one additive selected from salts or sugars, at least one preservative, or combinations thereof.

We claim:

1. A composition comprising an immobilized enzyme, wherein the immobilized enzyme comprises:
a. non-ordered amorphous silica having a silica surface that is functionalized with a methacryl group; and
b. a lipase immobilized on the silica surface that is functionalized with the methacryl group.

2. The composition of claim 1, wherein the immobilized enzyme comprises amorphous silica having a particle size ranging from 10 μm to 10 mm.

3. The composition of claim 1, wherein the immobilized enzyme comprises amorphous silica having an average pore diameter ranging from 10-150 nm.

4. The composition of claim 1, wherein the immobilized enzyme comprises amorphous silica having a surface area in the range of 10 m²/g to 500 m²/g.

5. The composition of claim 1, wherein the immobilized enzyme comprises amorphous silica having a pore volume ranging from 0.1 cc/g to 3.5 cc/g.

6. The composition of claim 1, wherein the immobilized enzyme comprises amorphous silica having a hydroxyl group concentration ranging from 0.1 OH/nm²-5 OH/nm².

7. The composition of claim 1, wherein the lipase Candida antartica lipase B (CalB) and their isoforms.

8. The composition of claim 1, further comprising at least one additive selected from salts, sugars, and surfactants, wherein the salts comprise calcium chloride, potassium chloride or combinations thereof, the sugars comprise glucose, fructose, sucrose, and combinations thereof.

9. The composition of claim 8, wherein the at least one additive selected from preservatives, alcohols and ethers, wherein the preservatives comprise potassium sorbate, sodium benzoate and combinations thereof, wherein the alcohol is selected from glycerol, glycol, polyethylene glycols, and polypropylene glycols and the ether is diethylene glycol butyl ether.

10. A method for immobilizing an enzyme,
the immobilized enzyme comprising:
non-ordered amorphous silica having a silica surface that is functionalized with a methacryl group; and
a lipase immobilized on the silica surface that is functionalized with the methacryl group,
the method comprising:
functionalizing a silica gel using an impregnation technique with a methacryl group precursor;
preparing a solution comprising the enzyme stabilized in a buffer;
optionally pretreating the functionalized silica; and
adding the solution to the functionalized silica and mixing to form an immobilized enzyme.

11. The method of claim 10, wherein the impregnation technique is selected from an incipient wetness impregnation technique and a slurry impregnation technique.

12. The method of claim 10, wherein the methacryl group precursor comprises at least one methacrylate group and at least one hydrolyzable silyl group, wherein the precursor is selected from silanes of the formula:
(methacryloyl-O—R¹)—(CH₂)ₙ—Si(R²)ₘ(OR³)₍₃₋ₘ₎
wherein:
n is an integer from 1 to 12;
m is 0, 1, or 2;
R¹ is a divalent alkylene group optionally substituted with one or more C₁-C₆ alkyl groups;
R² is a C₁-C₆ alkyl group or a halogen; and
R³ is independently selected from C₁-C₆ alkyl groups.

13. The method of claim 12, wherein m is 0 and R³ is independently selected from methyl or ethyl.

14. The method of claim 12, wherein n is 3.

15. The method of claim 12 wherein the methacryl group precursor is selected from the group consisting of:
(a) 3-(trimethoxysilyl)propyl methacrylate;
(b) 3-(triethoxysilyl)propyl methacrylate;
(c) 3-(methacryloyloxy)propyl methyldimethoxysilane;
(d) 3-(methacryloyloxy)propyl methyldiethoxysilane;
(e) 3-(methacryloyloxy)propyl dimethylchlorosilane;
(f) 6-(trimethoxysilyl)hexyl methacrylate; and
(g) 11-(trimethoxysilyl)undecyl methacrylate.

16. The method of claim 12, wherein the precursor is a trialkoxysilane selected from 3-(trimethoxysilyl)propyl methacrylate or 3-(triethoxysilyl)propyl methacrylate.

17. The method of claim 10, wherein silane used is in an amount to achieve an OH:silane molar ratio of 10 to 0.5.

18. The method of claim 10, wherein functionalizing the silica gel is done at room temperature.

19. The method of claim 10, wherein the buffer comprises sodium phosphate.

20. The method of claim 10, wherein the solution comprising the enzyme stabilized in a buffer has protein concentrations in the range of 0.5 mg/mL to 1.5 mg/mL.

21. The method of claim 10, wherein the silica is pretreated with an alcohol selected from methanol, ethanol, isopropanol, and n-propanol.

22. The method of claim 10, wherein the silica is pretreated with an ether, said ether is diethylene glycol butyl ether.

23. The method of claim 10, wherein the solution is added to the functionalized silica in an amount sufficient to achieve a functionalized silica:enzyme-buffer solution weight ratio in the range of 1:2 to 1:50.

24. The method of claim 10, wherein the solution is added to the functionalized silica in an amount sufficient to achieve a functionalized silica:enzyme-buffer solution weight ratio in the range of 1:10 to 1:30.

25. The method of claim 10, wherein once the solution is added to the functionalized silica mixing occurs at temperatures in the range of 4 °C to 60 °C for at least 1 hour, the method further comprising rinsing and drying steps after formation of the immobilized enzyme, wherein the rinsing step is done with deionized water and the drying in step occurs above room temperature.

26. The method of claim 25, wherein the rinsing step further comprises the use of one or more additives selected from salts, sugars, and surfactants,
wherein the salts comprise calcium chloride, potassium chloride or combinations thereof,
wherein the sugars comprise glucose, fructose, sucrose, and combinations thereof.

27. The method of claim 26, wherein the at least one additive selected from preservatives, alcohols and ethers, wherein the preservatives comprise potassium sorbate, sodium benzoate and combinations thereof, wherein the alcohol is selected from glycerol, glycol, polyethylene glycols, and polypropylene glycols.

28. The method of claim 27, wherein the ether is diethylene glycol butyl ether.

29. A method of performing an enzymatic reaction, the method comprising:
contacting a substrate with an immobilized enzyme comprising: non-ordered amorphous silica having a silica surface that is functionalized with a methacryl group; and a lipase immobilized on the silica surface that is functionalized with the methacryl group,
wherein contacting is performed under conditions effective to catalyze conversion of the substrate to a reaction product.

30. The method of claim 29, wherein the enzymatic reaction is a hydrolysis reaction or a transesterification reaction.

31. The method of claim 29, wherein contacting is performed at a temperature in a range of 0 °C to 120 °C and at a pH in a range of 5 to 9.

32. The method of claim 29, further comprising recovering the immobilized enzyme after the contacting and reusing the immobilized enzyme in at least 1 additional reaction cycles.
